(19) United States
(12) Patent Application Publication
(10) Pub. No.: US 2025/0380858 A1
(43) Pub. Date: Dec. 18, 2025

(54) MEDICAL MANIPULATOR SYSTEM, MEDICAL MANIPULATOR CONTROL METHOD, AND MEDICAL MANIPULATOR CONTROL DEVICE

(71) Applicant: OLYMPUS MEDICAL SYSTEMS CORP., Tokyo (JP)

(72) Inventors: Ryota YANAGAWA, Hachioji-shi (JP); Kosuke KISHI, Mitaka-shi (JP)

(73) Assignee: OLYMPUS MEDICAL SYSTEMS CORP., Tokyo (JP)

(21) Appl. No.: 19/050,207

(22) Filed: Feb. 11, 2025

Related U.S. Application Data
(60) Provisional application No. 63/660,691, filed on Jun. 17, 2024.

(51) Int. Cl. A61B 1/005; A61B 1/00

(52) U.S. Cl. CPC A61B 1/0057; A61B 1/00097

(57) ABSTRACT

A medical manipulator system includes a medical manipulator including a bending portion and a bending wire configured to bend the bending portion, an actuator configured to bend the bending portion by driving the bending wire, a sensor configured to detect tension of the bending wire, and a controller configured to control the actuator. The controller drives the bending wire so that the tension of the bending wire does not exceed a tension limit value determined on the basis of a traction quantity of the bending wire.

### CROSS-REFERENCE TO RELATED APPLICATIONS

Priority is claimed on U.S. Provisional Application No. 63/660,691, filed Jun. 17, 2024, the content of which is incorporated herein by reference.

### FIELD

The present disclosure relates to a medical manipulator system, a medical manipulator control method, and a medical manipulator control device.

### RELATED ART

Conventionally, medical manipulator systems are used for observation and treatment within a luminal organ such as an alimentary canal. In a medical manipulator system, an insertion portion or the like inserted into the luminal organ can be electrically driven. A user can control an operation for bending the insertion portion and the like from an extracorporeally arranged manipulation portion.

To widely observe the inside of the luminal organ, it is desirable to be able to drive the insertion portion or the like in large bending. On the other hand, when the insertion portion or the like is to be driven in large bending, because the insertion portion or the like in contact with the luminal organ exerts a large load on the luminal organ, or the insertion portion or the like fails due to excessive bending, it is desirable to limit the bending drive of the insertion portion or the like to a predetermined range.

Japanese Patent No. 3007699 (which is hereinafter referred to as Patent Document 1) describes an endoscope that limits the bending drive of the bending portion when the tension of a wire that bends the bending portion exceeds a predetermined value.

### SUMMARY

However, in the conventional medical manipulator system shown in Patent Document 1 and the like, because the bending drive of the insertion portion or the like is limited on the basis of the tension of the wire that bends the insertion portion or the like, the maximum bending angle of the insertion portion or the like decreases in a situation where the tension of the wire increases due to a factor other than the bending drive of the insertion portion or the like, for example, such as a situation where the wire meanders significantly.

Based on the above-described circumstances, an objective of the present disclosure is to provide a medical manipulator system, a medical manipulator control method, and a medical manipulator control device in which the bending drive of an insertion portion or the like is limited to a predetermined range and a maximum bending angle of the insertion portion or the like is unlikely to decrease.

According to a first aspect of the present disclosure, there is provided a medical manipulator system including: a medical manipulator including a bending portion and a bending wire configured to bend the bending portion; an actuator configured to bend the bending portion by driving the bending wire; a sensor configured to detect tension of the bending wire; and a controller configured to control the actuator, wherein the controller drives the bending wire so that the tension of the bending wire does not exceed a tension limit value determined on the basis of a traction quantity of the bending wire.

According to the medical manipulator system, the medical manipulator control method, and the medical manipulator control device of the present disclosure, the bending drive of an insertion portion or the like is limited to a predetermined range and a maximum bending angle of the insertion portion or the like is unlikely to decrease.

### BRIEF DESCRIPTION OF THE DRAWINGS

FIG. 1 is an overall view of a medical manipulator system according to a first embodiment.

FIG. 2 is a view showing an endoscope and a manipulation device of the medical manipulator system used by a practitioner.

FIG. 3 is a view showing an insertion portion of the endoscope.

FIG. 4 is a view showing a part of a bending portion of the insertion portion in a cross-sectional view.

FIG. 5 is an enlarged view of a joint ring of the bending portion in a region E shown in FIG. 4.

FIG. 6 is a cross-sectional view of the bending portion along line C1-C1 of FIGS. 4 and 5.

FIG. 7 is a view showing a first attachable portion before attachment to a drive device of the medical manipulator system.

FIG. 8 is a view showing a detachable upper/lower bending wire portion before attachment to the drive device.

FIG. 9 is a view showing the detachable upper/lower bending wire portion attached to the drive device.

FIG. 10 is a functional block diagram of the drive device.

FIG. 11 is a perspective view of the manipulation device of the medical manipulator system.

FIG. 12 is a functional block diagram of a video control device of the medical manipulator system.

FIG. 13 is a control flowchart of a drive controller of a control device of the medical manipulator system.

FIG. 14 is a diagram showing the insertion portion inserted into a large intestine.

FIG. 15 is a graph showing a traction quantity limit value and a tension limit value of the bending wire.

FIG. 16 is a graph showing a variation in the tension limit value.

FIG. 17 is a graph showing a variation in the traction quantity limit value.

### DETAILED DESCRIPTION

### First Embodiment

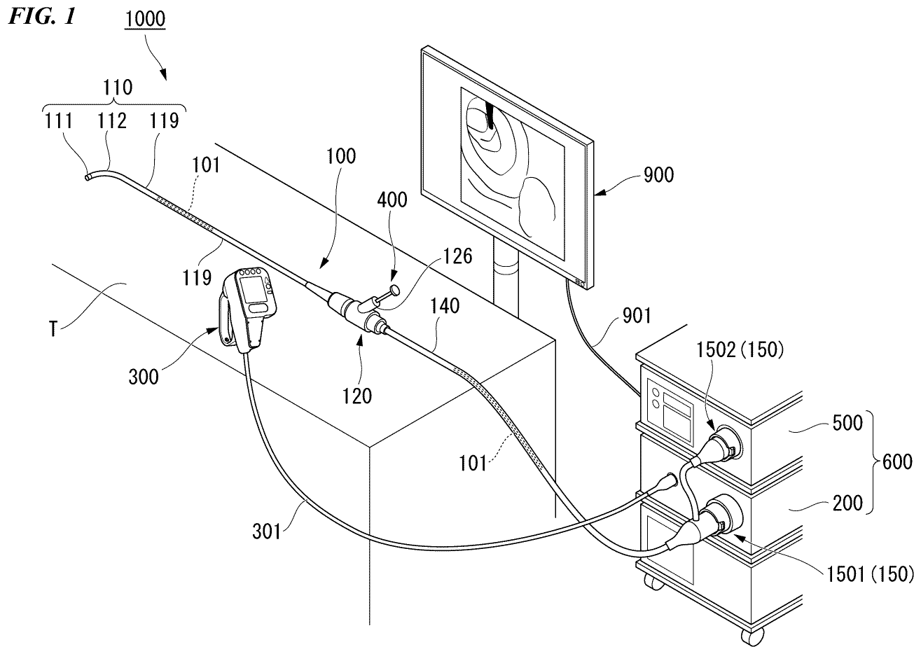

An electric endoscope system 1000 according to a first embodiment of the present disclosure will be described with reference to FIGS. 1 to 17. FIG. 1 is an overall view of the electric endoscope system 1000 according to the present embodiment. The electric endoscope system 1000 is an example of a medical manipulator system. Medical manipulators include electrically driven endoscopes, catheters, treatment tools, endoluminal devices, and the like to be intracorporeally inserted.

### Electric endoscope system 1000

The electric endoscope system 1000 is a medical system for observing and treating the inside of the body of a patient P lying on an operating table T, as shown in FIG. 1. The electric endoscope system 1000 includes an endoscope 100, a drive device 200, a manipulation device 300, a treatment tool 400, a video control device 500, and a display device 900.

The endoscope 100 is a device that is inserted into the lumen of the patient P to observe and treat an affected part. The endoscope 100 is detachable from the drive device 200. An internal path 101 is formed inside the endoscope 100. In the following description, a side of the endoscope 100 inserted into the lumen of the patient P is referred to as a “distal end side A1” and a side of the endoscope 100 attached to the drive device 200 is referred to as a “proximal end side A2.”

The drive device 200 is detachably connected to the endoscope 100 and the manipulation device 300. The drive device 200 electrically drives the endoscope 100 by driving a built-in motor on the basis of a manipulation input to the manipulation device 300. Moreover, the drive device 200 drives a built-in pump or the like on the basis of the manipulation input to the manipulation device 300 to cause the endoscope 100 to perform supplied air suction.

The manipulation device 300 is detachably connected to the drive device 200 via a manipulation cable 301. The manipulation device 300 may be able to communicate with the drive device 200 through wireless communication instead of wired communication. The practitioner S can electrically drive the endoscope 100 by manipulating the manipulation device 300.

The treatment tool 400 is a device that is inserted into the internal path 101 of the endoscope 100 and then inserted into the lumen of the patient P to treat the affected part. In FIG. 1, the treatment tool 400 is inserted into the internal path 101 of the endoscope 100 from a forceps port 126.

The video control device 500 is detachably connected to the endoscope 100 and acquires a captured image from the endoscope 100. The video control device 500 causes the display device 900 to display the captured image acquired from the endoscope 100 and a GUI image or a CG image for the purpose of providing information to a manipulator.

The drive device 200 and the video control device 500 constitute a control device 600 that controls the electric endoscope system 1000. The control device 600 may further include a peripheral device such as a video printer. The drive device 200 and the video control device 500 may be an integrated device.

The display device 900 is a device capable of displaying an image such as an LCD. The display device 900 is connected to the video control device 500 via a display cable 901.

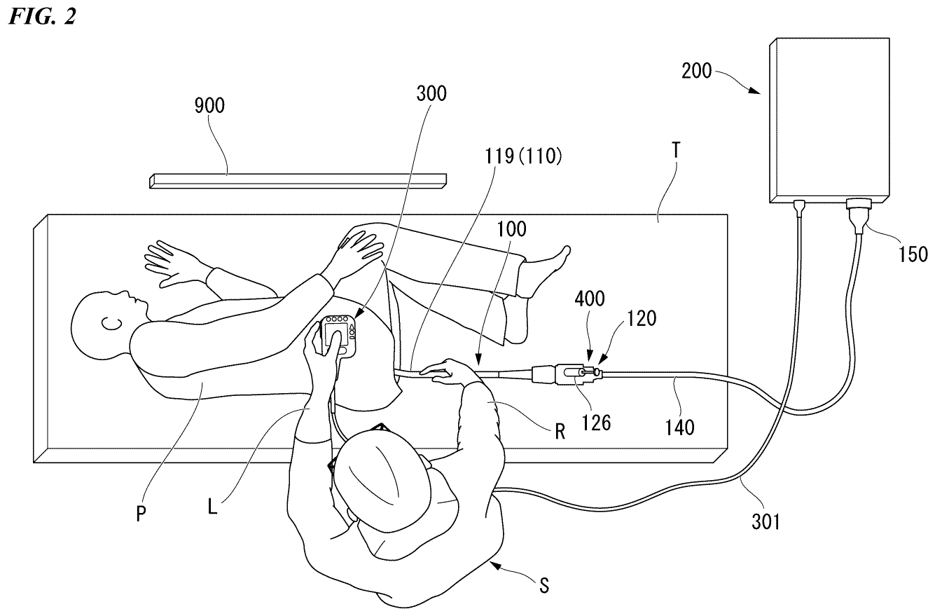

FIG. 2 is a view showing the endoscope 100 and the manipulation device 300 used by the practitioner S.

For example, the practitioner S manipulates the endoscope 100 inserted into the lumen from the anus of the patient P with a right hand R and manipulates the manipulation device 300 with a left hand L while observing the captured image displayed on the display device 900. Because the endoscope 100 and the manipulation device 300 are separated, the practitioner S can manipulate the endoscope 100 and the manipulation device 300 independently in a state in which the endoscope 100 and the manipulation device 300 do not affect each other.

### Endoscope 100

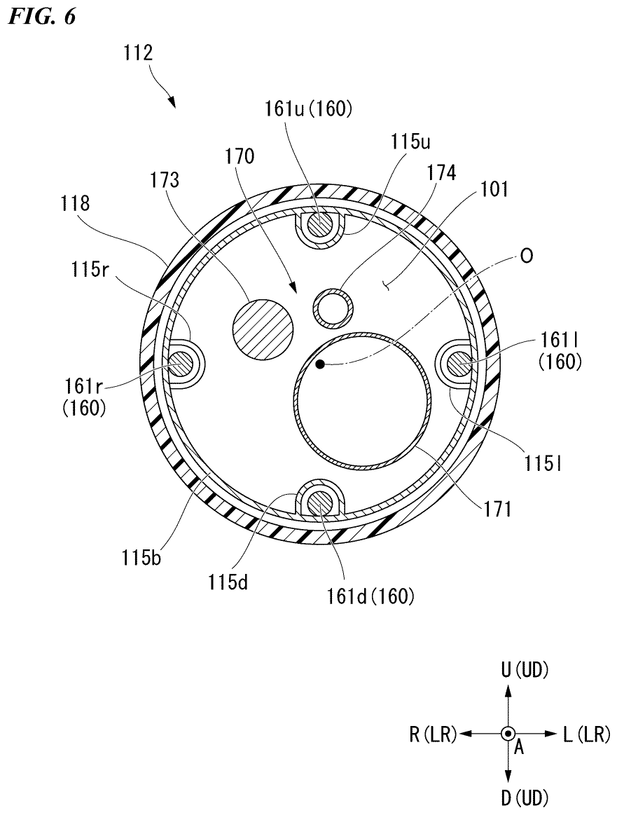

As shown in FIG. 1, the endoscope 100 includes an insertion portion 110, a connection portion 120, an extracorporeal flexible portion 140, a detachable portion 150, a bending wire 160 (see FIG. 6), and a built-in object 170 (see FIG. 6). The insertion portion 110, the connection portion 120, the extracorporeal flexible portion 140, and the detachable portion 150 are connected in order from the distal end side.

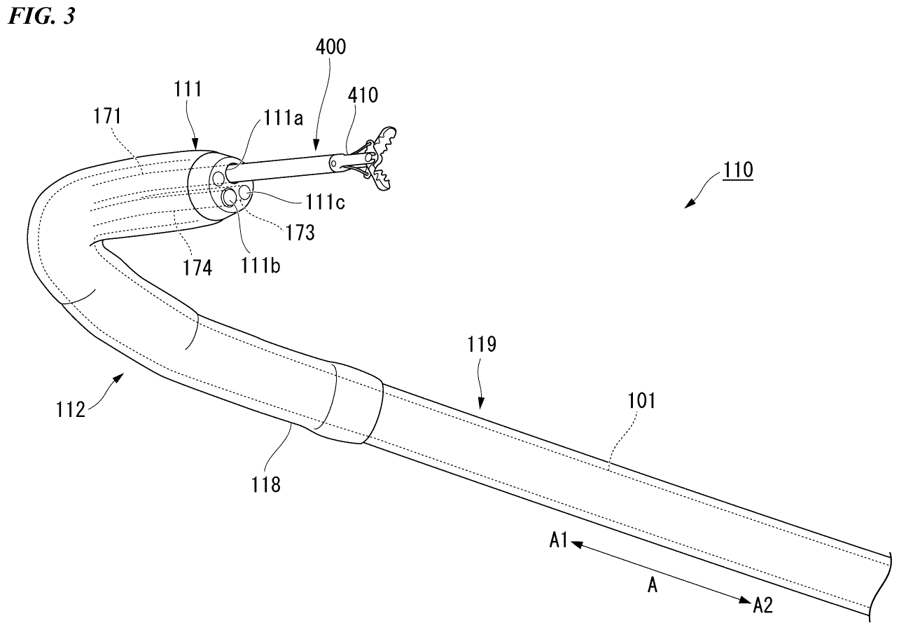

FIG. 3 is a view showing the insertion portion 110 of the endoscope 100.

Within the endoscope 100, the internal path 101 extending in a longitudinal direction A of the endoscope 100 is formed from the distal end of the insertion portion 110 to the proximal end of the detachable portion 150. The bending wire 160 and the built-in object 170 are inserted into the internal path 101.

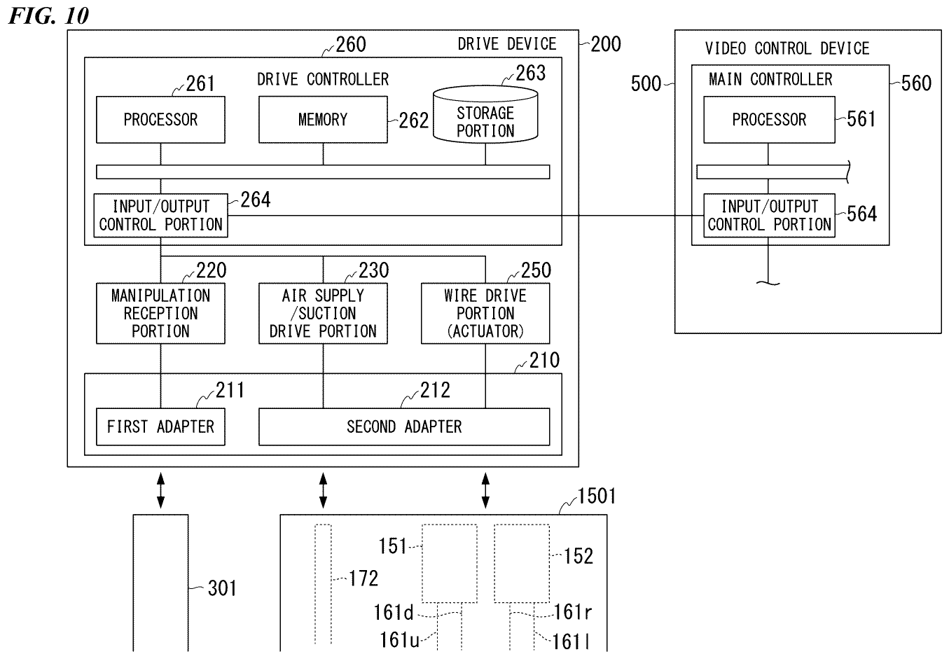

The built-in object 170 includes a channel tube 171, an air supply/suction tube 172 (see FIG. 10), an imaging cable 173, and a light guide 174.

### Insertion Portion 110

The insertion portion 110 is an elongated long member that can be inserted into the lumen. The insertion portion 110 includes a distal end portion 111, a bending portion 112, and an intracorporeal flexible portion 119. The distal end portion 111, the bending portion 112, and the intracorporeal flexible portion 119 are connected in order from the distal end side.

As shown in FIG. 3, the distal end portion 111 includes an opening 111a, an illumination portion 111b, and an imaging portion 111c. The opening 111a is an opening that communicates with the channel tube 171. As shown in FIG. 3, a treatment portion 410 such as a gripping forceps provided at the distal end of the treatment tool 400 into which the channel tube 171 is inserted is protruded from or recessed into the opening 111a.

The illumination portion 111b is connected to the light guide 174 that guides the illumination light, and emits illumination light that illuminates an imaging target. The imaging portion 111c includes an image sensor such as a CMOS and captures an imaging target. An imaging signal is sent to the video control device 500 via the imaging cable 173.

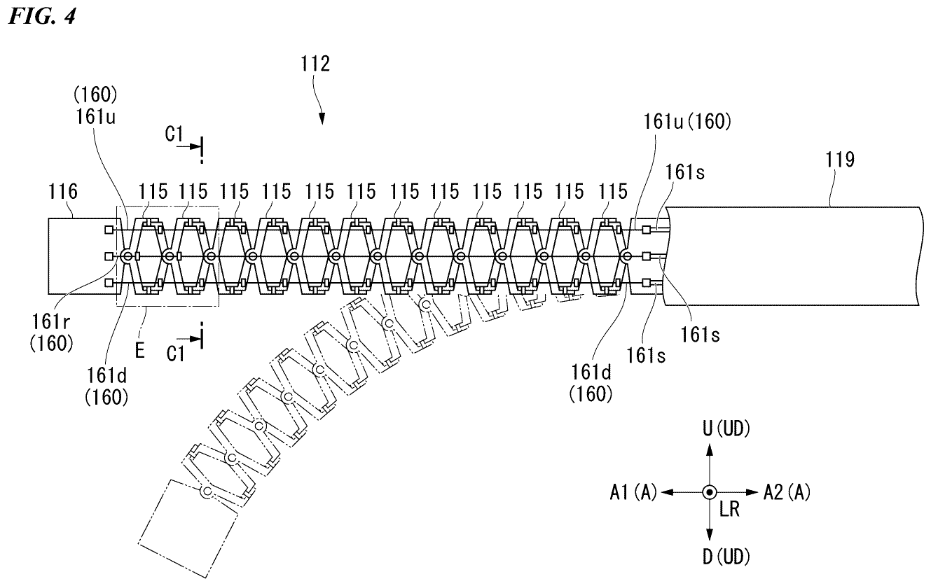

FIG. 4 is a view showing a part of the bending portion 112 in a cross-sectional view.

The bending portion 112 includes a plurality of joint rings (also referred to as bending pieces) 115, a distal end portion 116 connected to the distal ends of the plurality of joint rings 115, and an outer sheath 118 (see FIG. 3). The plurality of joint rings 115 and the distal end portion 116 are connected in the longitudinal direction A inside the outer sheath 118. In addition, the shapes and number of joint rings 115 provided in the bending portion 112 are not limited to those shown in FIG. 4.

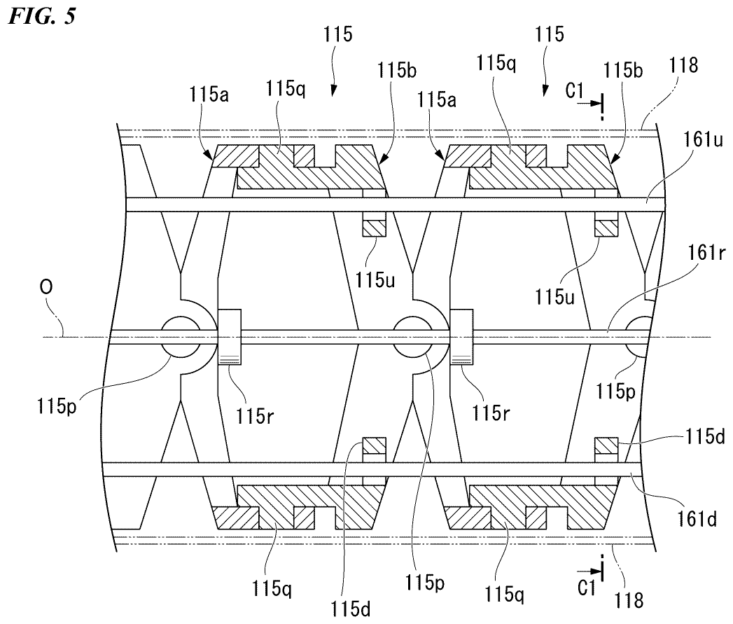

FIG. 5 is an enlarged view of the joint ring 115 in a region E shown in FIG. 4.

The joint ring 115 is a short cylindrical member formed of a metal. The plurality of joint rings 115 are connected so that the internal spaces of the adjacent joint rings 115 become continuous spaces.

The joint ring 115 has a first joint ring 115a on the distal end side and a second joint ring 115b on the proximal end side. The first joint ring 115a and the second joint ring 115b are rotatably connected by a first rotation pin 115p in a vertical direction (also referred to as a “UD direction”) perpendicular to the longitudinal direction A.

In the adjacent joint rings 115, the second joint ring 115b in the joint ring 115 on the distal end side and the first joint ring 115a in the joint ring 115 on the proximal end side are rotatably connected by a second rotation pin 115q in a left/right direction (an “LR direction”) perpendicular to the longitudinal direction A and the UD direction.

The first joint ring 115a and the second joint ring 115b are alternately connected by the first rotation pin 115p and the second rotation pin 115q, and the bending portion 112 is freely bent in a desired direction.

FIG. 6 is a cross-sectional view of the bending portion 112 along line C1-C1 of FIGS. 4 and 5.

On the inner circumferential surface of the second joint ring 115b, the upper wire guide 115u and the lower wire guide 115d are formed. The upper wire guide 115u and the lower wire guide 115d are arranged on both sides between which a central axis O in the longitudinal direction A is sandwiched in the UD direction. On the inner circumferential surface of the first joint ring 115a, a left wire guide 115l and a right wire guide 115r are formed. The left wire guide 115l and the right wire guide 115r are arranged on both sides between which the central axis O in the longitudinal direction A is sandwiched in the LR direction.

On the upper wire guide 115u, the lower wire guide 115d, the left wire guide 115l, and the right wire guide 115r, a through-hole into which the bending wire 160 is inserted is formed in the longitudinal direction A.

The bending wire 160 is a wire for bending the bending portion 112. The bending wire 160 extends to the detachable portion 150 through the internal path 101. As shown in FIGS. 4 and 6, the bending wire 160 includes an upper bending wire 161u, a lower bending wire 161d, a left bending wire 1611, a right bending wire 161r, and four wire sheaths 161s.

As shown in FIG. 4, the upper bending wire 161u, the lower bending wire 161d, the left bending wire 161l, and the right bending wire 161r are inserted into the wire sheaths 161s. The distal end of the wire sheath 161s is attached to the joint ring 115 at the proximal end of the bending portion 112. The wire sheath 161s extends to the detachable portion 150.

The upper bending wire 161u and the lower bending wire 161d are wires for bending the bending portion 112 in the UD direction. The upper bending wire 161u is inserted into the upper wire guide 115u. The lower bending wire 161d is inserted into the lower wire guide 115d.

The distal ends of the upper bending wire 161u and the lower bending wire 161d are fixed to the distal end portion 116 of the distal end of the bending portion 112 as shown in FIG. 4. The distal ends of the upper bending wire 161u and the lower bending wire 161d fixed to the distal end portion 116 are arranged on both sides between which the central axis O in the longitudinal direction A is sandwiched in the UD direction.

The left bending wire 161l and the right bending wire 161r are wires for bending the bending portion 112 in the LR direction. The left bending wire 161l is inserted into the left wire guide 115l. The right bending wire 161r is inserted into the right wire guide 115r.

The distal ends of the left bending wire 161l and the right bending wire 161r are fixed to the distal end portion 116 of the bending portion 112 as shown in FIG. 4. The distal ends of the left bending wire 161l and the right bending wire 161r fixed to the distal end portion 116 are arranged on both sides between which the central axis O in the longitudinal direction A is sandwiched in the LR direction.

The bending portion 112 is freely bent in a desired direction by pulling or relaxing the bending wires 160 (the upper bending wire 161u, the lower bending wire 161d, the left bending wire 161l, and the right bending wire 161r).

As shown in FIG. 6, the bending wire 160, the channel tube 171, the imaging cable 173, and the light guide 174 are inserted into the internal path 101 formed inside the bending portion 112.

The intracorporeal flexible portion 119 is a long and flexible tubular member. The bending wire 160, the channel tube 171, the imaging cable 173, and the light guide 174 are inserted into the internal path 101 formed in the intracorporeal flexible portion 119.

### Connection Portion 120

As shown in FIG. 1, the connection portion 120 is a member that connects the intracorporeal flexible portion 119 and the extracorporeal flexible portion 140 of the insertion portion 110. The connection portion 120 includes the forceps port 126 that is an insertion port into which the treatment tool 400 is inserted.

### Extracorporeal Flexible Portion 140

The extracorporeal flexible portion 140 is a long tubular member. The bending wire 160, the imaging cable 173, the light guide 174, and the air supply/suction tube 172 (see FIG. 10) are inserted into the internal path 101 formed inside the extracorporeal flexible portion 140.

### Detachable Portion 150

As shown in FIG. 1, the detachable portion 150 includes a first detachable portion 1501 attached to the drive device 200 and a second detachable portion 1502 attached to the video control device 500. In addition, the first detachable portion 1501 and the second detachable portion 1502 may be an integrated detachable portion.

The internal path 101 formed inside the extracorporeal flexible portion 140 branches into the first detachable portion 1501 and the second detachable portion 1502. The bending wire 160, and the air supply/suction tube 172 are inserted into the first detachable portion 1501. The imaging cable 173 and the light guide 174 are inserted into the second detachable portion 1502.

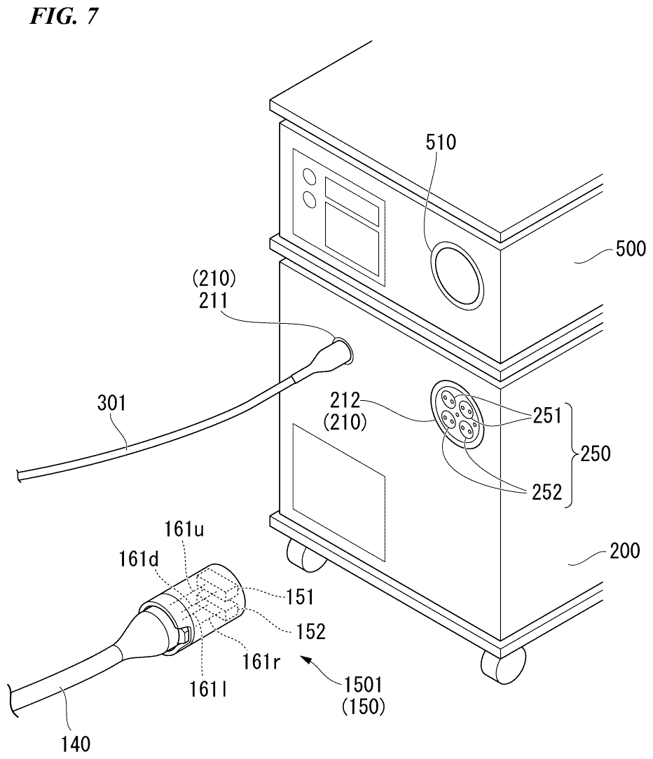

FIG. 7 is a view showing the first detachable portion 1501 before attachment to the drive device 200.

The first detachable portion 1501 includes a detachable upper/lower bending wire portion 151 and a detachable left/right bending wire portion 152.

The detachable upper/lower bending wire portion 151 is a mechanism for detachably connecting wires (the upper bending wire 161u and the lower bending wire 161d) for bending the bending portion 112 in the UD direction to the drive device 200.

The detachable left/right bending wire portion 152 is a mechanism for detachably connecting wires (the left bending wire 161l and the right bending wire 161r) for bending the bending portion 112 in the LR direction to the drive device 200.

Because the detachable left/right bending wire portion 152 has a structure equivalent to that of the detachable upper/lower bending wire portion 151, illustration and description thereof are omitted.

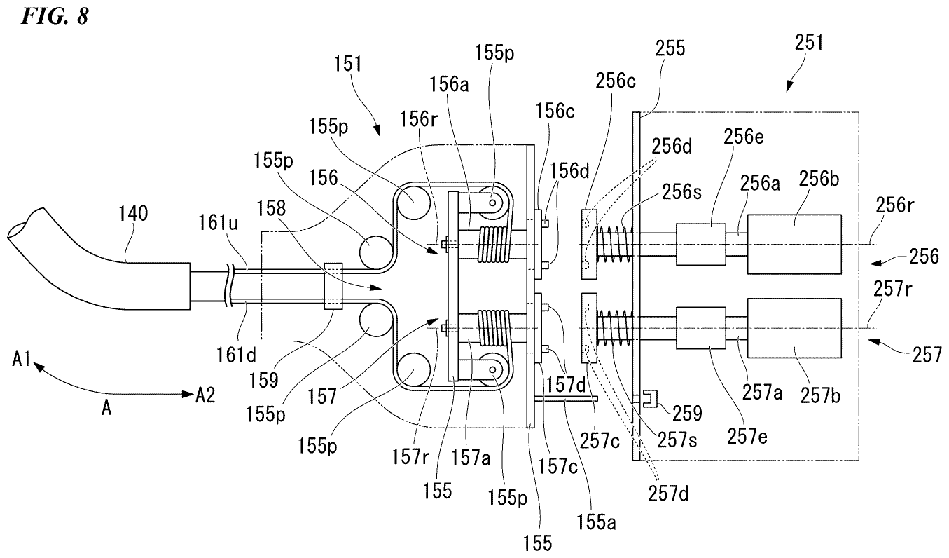

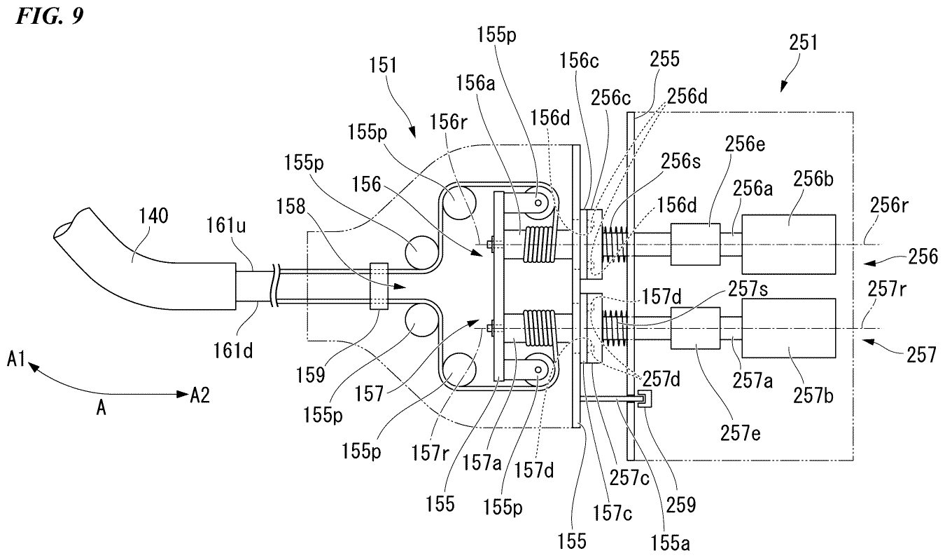

FIG. 8 is a view showing the detachable upper/lower bending wire portion 151 before attachment to the drive device 200. FIG. 9 is a view showing the detachable upper/lower bending wire portion 151 attached to the drive device 200. The detachable upper/lower bending wire portion 151 includes a support member 155, a first rotation drum 156, a second rotation drum 157, and a tension sensor 159.

The support member 155 supports the first rotation drum 156, the second rotation drum 157, and a connection member 158. The support member 155 includes an attachment/detachment detection dog 155a arranged on the proximal end side of the detachable upper/lower bending wire portion 151, and a plurality of bend pulleys 155p.

The bend pulley 155p changes a transport direction of the upper bending wire 161u inserted into the extracorporeal flexible portion 140 and guides the upper bending wire 161u to the first rotation drum 156. Moreover, the bend pulley 155p changes the transport direction of the lower bending wire 161d inserted into the extracorporeal flexible portion 140 and guides the lower bending wire 161d to the second rotation drum 157.

The first rotation drum 156 is supported by the support member 155 so that the first rotation drum 156 can be rotated around the first drum rotation axis 156r extending in a longitudinal direction A. The first rotation drum 156 has a first winding pulley 156a and a first coupling portion 156c.

The first winding pulley 156a pulls or sends the upper bending wire 161u by rotating around the first drum rotation axis 156r. By rotating the first winding pulley 156a clockwise from the distal end side to the proximal end side, the upper bending wire 161u is wound around the first winding pulley 156a and pulled. In contrast, the first winding pulley 156a rotates counterclockwise, and therefore the upper bending wire 161u is sent from the first winding pulley 156a. With this configuration, even if an amount of forward/rearward movement of the upper bending wire 161u is large, a pulled portion is compactly stored and does not take up space.

The first coupling portion 156c is a disc member that rotates around the first drum rotation axis 156r. The first coupling portion 156c is fixed to the proximal end of the first winding pulley 156a and rotates integrally with the first winding pulley 156a. The first coupling portion 156c is exposed on the proximal end side of the detachable upper/lower bending wire portion 151. Two first fitting convex portions 156d are formed on the surface of the proximal end side of the first coupling portion 156c. The two first fitting convex portions 156d are formed on both sides between which the first drum rotation axis 156r is sandwiched.

The second rotation drum 157 is supported by the support member 155 so that the second rotation drum 157 can be rotated around the second drum rotation axis 157r extending in the longitudinal direction A. The second rotation drum 157 includes a second winding pulley 157a and a second coupling portion 157c.

The second winding pulley 157a pulls or sends the lower bending wire 161d by rotating around the second drum rotation axis 157r. By rotating the second winding pulley 157a counterclockwise from the distal end side to the proximal end side, the lower bending wire 161d is wound around the second winding pulley 157a and pulled. In contrast, the second winding pulley 157a rotates clockwise, and therefore the lower bending wire 161d is sent from the second winding pulley 157a.

The second coupling portion 157c is a disc member that rotates around the second drum rotation axis 157r. The second coupling portion 157c is fixed to the proximal end of the second winding pulley 157a and rotates integrally with the second winding pulley 157a. The second coupling portion 157c is exposed on the proximal end side of the detachable upper/lower bending wire portion 151. Two second fitting convex portions 157d are formed on the surface of the proximal end side of the second coupling portion 157c. The two second fitting convex portions 157d are formed on both sides between which the second drum rotation axis 157r is sandwiched.

The tension sensor 159 detects the tension of the upper bending wire 161u and the lower bending wire 161d. A detection result of the tension sensor 159 is acquired by the drive controller 260.

### Drive Device 200

FIG. 10 is a functional block diagram of the drive device 200.

The drive device 200 includes an adapter 210, a manipulation reception portion 220, an air supply/suction drive portion 230, a wire drive portion (actuator) 250, and the drive controller 260.

The adapter 210 has a first adapter 211 and a second adapter 212, as shown in FIG. 7. The first adapter 211 is an adapter to which the manipulation cable 301 is detachably connected. The second adapter 212 is an adapter to which the first detachable portion 1501 of the endoscope 100 is detachably connected.

The manipulation reception portion 220 receives a manipulation input from the manipulation device 300 via the manipulation cable 301. When the manipulation device 300 and the drive device 200 perform communication according to wireless communication instead of wired communication, the manipulation reception portion 220 has a known wireless reception module.

The air supply/suction drive portion 230 is connected to the air supply/suction tube 172 inserted into the internal path 101 of the endoscope 100. The air supply/suction drive portion 230 includes a pump or the like and supplies air to the air supply/suction tube 172. Moreover, the air supply/suction drive portion 230 suctions air from the air supply/suction tube 172.

The wire drive portion (actuator) 250 is coupled with the detachable upper/lower bending wire portion 151 and the detachable left/right bending wire portion 152 to drive the bending wire 160.

As shown in FIG. 7, the wire drive portion 250 includes an upper/lower bending wire drive portion (first actuator) 251 and a left/right bending wire drive portion (second actuator) 252.

The upper/lower bending wire drive portion 251 is a mechanism that is coupled with the detachable upper/lower bending wire portion 151 to drive wires (the upper bending wire 161u and the lower bending wire 161d) that bend the bending portion 112 in the UD direction.

The left/right bending wire drive portion 252 is a mechanism that is coupled with the detachable left/right bending wire portion 152 to drive wires (the left bending wire 161l and the right bending wire 161r) that bends the bending portion 112 in the LR direction.

Because the left/right bending wire drive portion 252 has a structure equivalent to that of the upper/lower bending wire drive portion 251, illustration and description thereof are omitted.

As shown in FIG. 8, the upper/lower bending wire drive portion 251 includes a support member 255, an upper bending wire drive portion 256, a lower bending wire drive portion 257, and a detachable sensor 259.

The upper bending wire drive portion 256 is coupled with the first rotation drum 156 of the detachable upper/lower bending wire portion 151 to drive the upper bending wire 161u. The upper bending wire drive portion 256 includes a first shaft 256a, a first motor portion 256b, a first coupled portion 256c, a first torque sensor 256e, and a first elastic member 256s.

The first shaft 256a is supported by the support member 255 so that the first shaft 256a can rotate around a first shaft rotation axis 256r and move forward and rearward in the longitudinal direction A. When the first detachable portion 1501 of the endoscope 100 is attached to the drive device 200, the first shaft rotation axis 256r coincides with the first drum rotation axis 156r.

The first motor portion 256b includes a first motor such as a DC motor, a first motor driver that drives the first motor, and a first motor encoder. The first motor rotates the first shaft 256a around the first shaft rotation axis 256r. The first motor driver is controlled by the drive controller 260.

The first coupled portion 256c is a disc member that rotates around the first shaft rotation axis 256r. The first coupled portion 256c is fixed to the distal end of the first shaft 256a and rotates integrally with the first shaft 256a. As shown in FIG. 8, the first coupled portion 256c is exposed on the distal end side of the upper/lower bending wire drive portion 251. Two first fitting concave portions 256d are formed on the surface of the distal end side of the first coupled portion 256c. The two first fitting concave portions 256d are formed on both sides between which the first shaft rotation axis 256r is sandwiched.

As shown in FIG. 9, the first fitting convex portion 156d and the first fitting concave portion 256d are fitted and the first coupling portion 156c and the first coupled portion 256c are coupled. As a result, the rotation of the first shaft 256a by the first motor portion 256b is transmitted to the first rotation drum 156. The upper bending wire 161u is pulled by rotating the first shaft 256a clockwise from the distal end side to the proximal end side. In contrast, the first shaft 256a rotates counterclockwise, and therefore the upper bending wire 161u is sent.

The first torque sensor 256e detects rotational torque centered on the first shaft rotation axis 256r of the first shaft 256a. A detection result of the first torque sensor 256e is acquired by the drive controller 260.

The first elastic member 256s is, for example, a compression spring, and has a distal end portion in contact with the first coupled portion 256c and a proximal end portion in contact with the support member 255. The first elastic member 256s biases the first coupled portion 256c to the distal end side Al. As shown in FIG. 9, when the first coupling portion 156c is attached, the first coupled portion 256c moves to the proximal end side A2 together with the first shaft 256a.

The lower bending wire drive portion 257 is coupled with the second rotation drum 157 of the detachable upper/lower bending wire portion 151 to drive the lower bending wire 161d. The lower bending wire drive portion 257 includes the second shaft 257a, the second motor portion 257b, the second coupled portion 257c, the second torque sensor 257e, and the second elastic member 257s.

The second shaft 257a is supported by the support member 255 so that second shaft 257a can rotate around the second shaft rotation axis 257r and can move forward and rearward in the longitudinal direction A. When the first detachable portion 1501 of the endoscope 100 is attached to the drive device 200, the second shaft rotation axis 257r coincides with the second drum rotation axis 157r.

The second motor portion 257b includes a second motor such as a DC motor, a second motor driver that drives the second motor, and a second motor encoder. The second motor rotates the second shaft 257a around the second shaft rotation axis 257r. The second motor driver is controlled by the drive controller 260.

The second coupled portion 257c is a disc member that rotates around the second shaft rotation axis 252r. The second coupled portion 257c is fixed to the distal end of the second shaft 257a and rotates integrally with the second shaft 257a. As shown in FIG. 8, the second coupled portion 257c is exposed on the distal end side of the upper/lower bending wire drive portion 251. Two second fitting concave portions 257d are formed on the surface of the distal end side of the second coupled portion 257c. The two second fitting concave portions 257d are formed on both sides between which the second shaft rotation axis 257r is sandwiched.

As shown in FIG. 9, the second fitting convex portion 157d and the second fitting concave portion 257d are fitted and the second coupling portion 157c and the second coupled portion 257c are coupled. As a result, the rotation of the second shaft 257a by the second motor portion 257b is transmitted to the second rotation drum 157. The second shaft 257a rotates counterclockwise when viewed from the distal end side to the proximal end side, and therefore the lower bending wire 161d is pulled. In contrast, the second shaft 257a rotates clockwise, and therefore the lower bending wire 161d is sent.

The second torque sensor 257e detects rotational torque centered on the second shaft rotation axis 257r of the second shaft 257a. A detection result of the second torque sensor 257e is acquired by the drive controller 260.

The second elastic member 257s is, for example, a compression spring, and has a distal end portion in contact with the second coupled portion 257c and a proximal end portion in contact with the support member 255. The second elastic member 257s biases the second coupled portion 257c to the distal end side Al. As shown in FIG. 9, when the second coupling portion 157c is attached, the second coupled portion 257c moves to the proximal end side A2 together with the second shaft 257a.

As shown in FIG. 9, the detachable sensor 259 detects the attachment and detachment of the detachable upper/lower bending wire portion 151 for the upper/lower bending wire drive portion 251 by detecting the engagement and non-engagement with the attachment/detachment detection dog 155a. A detection result of the detachable sensor 259 is acquired by the drive controller 260.

According to the above-described mechanism, when the detachable upper/lower bending wire portion 151 is attached to the upper/lower bending wire drive portion 251, the upper bending wire drive portion 256 can independently drive the upper bending wire 161u and the lower bending wire drive portion 257 can independently drive the lower bending wire 161d. Therefore, even if a distance from the bending portion 112 of the endoscope 100 to the drive device 200 is longer than that of a conventional flexible endoscope, a bending manipulation on the bending portion 112 can be controlled with high accuracy.

The drive controller 260 controls the entire drive device 200. The drive controller 260 acquires a manipulation input received by the manipulation reception portion 220. The drive controller 260 controls the air supply/suction drive portion 230 and the wire drive portion 250 on the basis of the acquired manipulation input.

The drive controller 260 is a computer capable of executing a program. The computer includes a processor 261, a memory 262, a storage portion 263 capable of storing programs and data, and an input/output control portion 264. Functions of the drive controller 260 are implemented by the processor executing the program. At least some of the functions of the drive controller 260 may be implemented by a dedicated logic circuit.

Because the drive controller 260 controls a plurality of motors that drive a plurality of bending wires 160 with high accuracy, the drive controller 260 desirably has high calculation performance.

In addition, the drive controller 260 may further include constituent elements other than the processor 261, the memory 262, the storage portion 263, and the input/output control portion 264. For example, the drive controller 260 may further include an image calculation portion that performs a part or all of image processing and image recognition processing. The image calculation portion is further included, and therefore the drive controller 260 can execute specific image processing and image recognition processing at a high speed. The image calculation portion may be mounted in a separate hardware device connected by a communication circuit.

### Manipulation Device 300

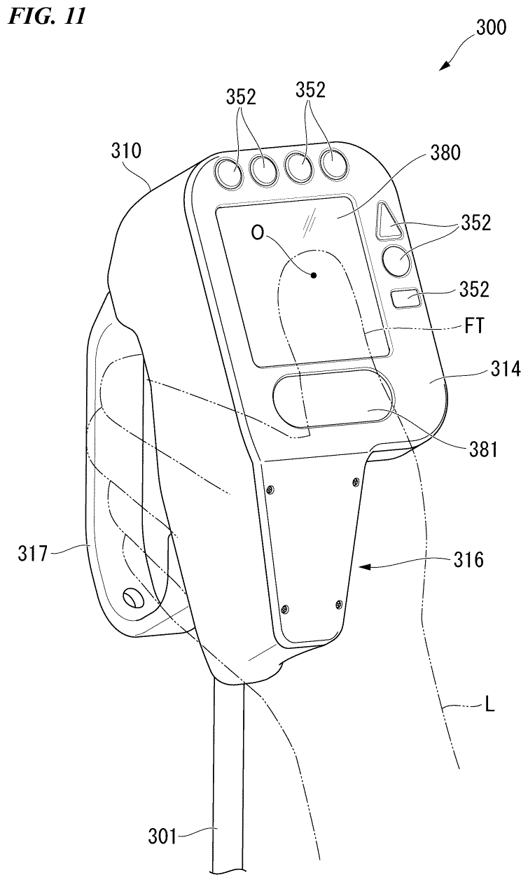

FIG. 11 is a perspective view of the manipulation device 300.

The manipulation device 300 is a device to which a manipulation for driving the endoscope 100 is input. The input manipulation input is transmitted to the drive device 200 via the manipulation cable 301. The manipulation device 300 may be able to communicate with the drive device 200 according to wireless communication instead of wired communication.

The manipulation device 300 includes a manipulation portion body 310, an air supply button, a suction button, various buttons 352, a touchpad 380, and a touch sensor 381.

The manipulation portion body 310 is formed in an approximately bar shape that can be held by the practitioner S with the left hand L. The manipulation portion body 310 includes a touchpad support portion 314 provided upward, a grip portion 316 provided downward, and a handle 317 provided rearward. As shown in FIG. 11, the practitioner S can manipulate the touchpad 380 with a thumb finger FT of the left hand L while gripping the grip portion 316 with the left hand L.

The touchpad 380 is a touch-sensitive interface to which a bending manipulation or the like on the bending portion 112 is input. The touchpad 380 may be a touch panel.

### Video Control Device 500

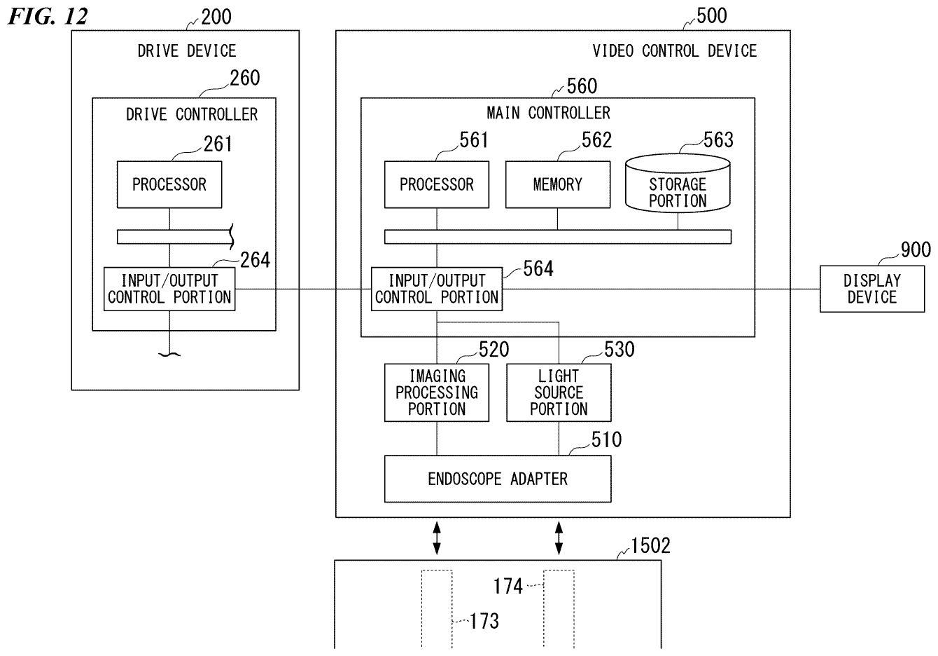

FIG. 12 is a functional block diagram of the video control device 500.

The video control device 500 controls the electric endoscope system 1000. The video control device 500 includes a third adapter 510, an imaging processing portion 520, a light source portion 530, and a main controller 560.

The third adapter 510 is an adapter to which the second detachable portion 1502 of the endoscope 100 is detachably connected.

The imaging processing portion 520 converts an imaging signal acquired from the imaging portion 111c of the distal end portion 111 into a captured image via the imaging cable 173.

The light source portion 530 generates illumination light radiated to an imaging target. The illumination light generated by the light source portion 530 is guided to the illumination portion 111b of the distal end portion 111 via the light guide 174.

The main controller 560 is a computer capable of executing a program. The computer includes a processor 561, a memory 562, a storage portion 563 capable of storing programs and data, and an input/output control portion 564. Functions of the main controller 560 are implemented by the processor 561 executing a program. At least some of the functions of the main controller 560 may be implemented by a dedicated logic circuit.

The main controller 560 includes the processor 561, the memory 562 from which a program can be read, the storage portion 563, and the input/output control portion 564.

The storage portion 563 is a non-volatile recording medium that stores the above-described programs and necessary data. The storage portion 563 includes, for example, a ROM, a hard disk, or the like. The program recorded in the storage portion 563 is read into the memory 562 and executed by the processor 561.

The input/output control portion 564 is connected to an imaging processing portion 520, a light source portion 530, a drive device 200, a display device 900, an input device (not shown), and a network device (not shown). The input/output control portion 564 performs the transmission/reception of data and/or the transmission/reception of control signals for the connected device on the basis of the control of the processor 561.

The main controller 560 can perform image processing on the captured image acquired by the imaging processing portion 520. The main controller 560 can generate a GUI image or a CG image for the purpose of providing information to the practitioner S. The main controller 560 can cause the display device 900 to display a captured image, a GUI image, or a CG image.

The main controller 560 is not limited to an integrated hardware device. For example, the main controller 560 may be configured by separating its part as a separate hardware device and then connecting the separated hardware device with a communication circuit. For example, the main controller 560 may be a cloud system in which the separated storage portion 563 is connected by the communication circuit.

The main controller 560 may further include constituent elements other than the processor 561, the memory 562, the storage portion 563, and the input/output control portion 564 shown in FIG. 12. For example, the main controller 560 may further include an image calculation portion that performs a part or all of the image processing and image recognition processing performed by the processor 561. The image calculation portion is further provided, and therefore the main controller 560 can execute specific image processing and image recognition processing at a high speed. The image calculation portion may be mounted in a separate hardware device connected by the communication circuit.

### Operation of Electric Endoscope System 1000

Next, an operation of the electric endoscope system 1000 of the present embodiment will be described. Specifically, a procedure for observing and treating the affected area formed on the tube wall in the large intestine using the electric endoscope system 1000 will be described.

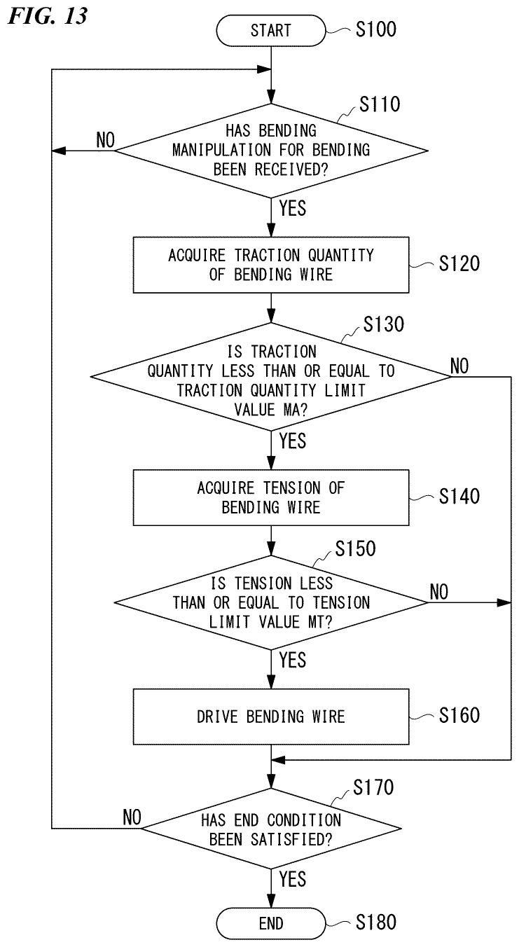

Hereinafter, description will be given according to a control flowchart of the drive controller 260 of the control device 600 shown in FIG. 13. When the control device 600 is activated, the drive controller 260 starts bending drive control of the bending wire 160 after initialization is performed (step S100). Subsequently, the drive controller 260 (mainly, the processor 261) executes step S110.

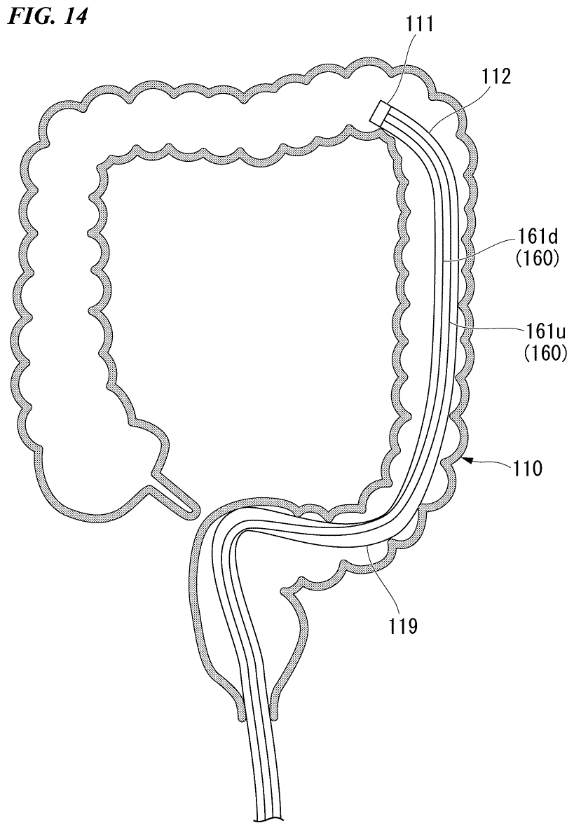

FIG. 14 is a diagram showing the insertion portion 110 inserted into the large intestine.

The practitioner S inserts the insertion portion 110 of the endoscope 100 into the large intestine from the anus of the patient P. While observing the imaging image displayed on the display device 900, the practitioner S moves the insertion portion 110 and causes the distal end portion 111 to be in proximity to the affected area while manipulating the intracorporeal flexible portion 119 in the body with the right hand R. Moreover, the practitioner S manipulates the manipulation device 300 with the left hand L and inputs a bending manipulation on the bending portion 112.

### Step S110

In step S110, the drive controller 260 acquires a bending manipulation on the bending portion 112 input to the touchpad 380 of the manipulation device 300.

### Step S120

The drive controller 260 acquires a traction quantity of the bending wire 160 in step S120. The drive controller 260 acquires the traction quantity of the bending wire 160 on the basis of a rotation quantity (drive quantity) in the shaft rotation axis (drive axis) from the first motor encoder of the first motor portion 256b, the second motor encoder of the second motor portion 257b, or the like. In addition, the drive controller 260 may acquire the traction quantity of the bending wire 160 by calculating the traction quantity of the bending wire 160 from a drive history of the bending wire 160. Subsequently, the drive controller 260 executes step S130.

### Step S130

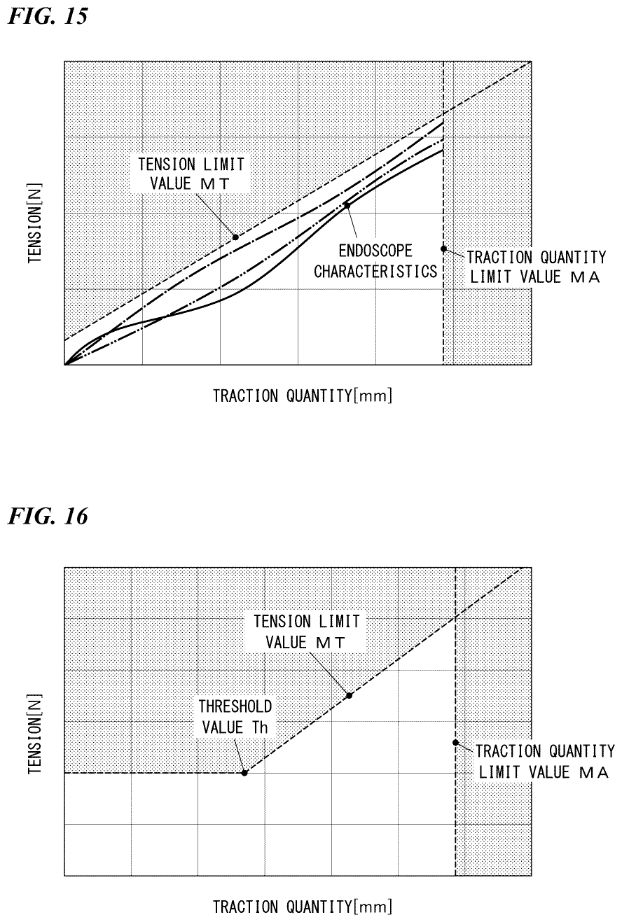

FIG. 15 is a graph showing a traction quantity limit value MA and a tension limit value MT of the bending wire 160. In step S130, the drive controller 260 calculates the traction quantity of the bending wire 160 when the bending wire 160 is driven on the basis of the received bending manipulation using the acquired traction quantity of the bending wire 160 as a reference. The drive controller 260 compares the traction quantity of the bending wire 160 when the bending wire 160 is driven on the basis of the received bending manipulation with a predetermined traction quantity limit value MA.

The “traction quantity limit value MA” is a maximum value of the traction quantity for pulling the bending wire 160 regardless of a bending shape of the flexible portion (the portion including the insertion portion 110 and the extracorporeal flexible portion 140). The “traction quantity limit value MA” is, for example, a value corresponding to the maximum value of the bending angle of the bending portion 112. The “traction quantity limit value MA” can be set to a value corresponding to the limit of the traction quantity in which the joint ring (bending piece) 115 does not deform, for example, in consideration of, for example, the durability of the joint ring (bending piece) 115 of the bending portion 112.

When the traction quantity of the bending wire 160 in the case where the bending wire 160 is driven on the basis of the received bending manipulation exceeds the traction quantity limit value MA, the drive controller 260 does not drive the bending wire 160 and executes step S170. That is, the drive controller 260 drives the bending wire 160 so that the traction quantity of the bending wire 160 does not exceed the traction quantity limit value MA.

The drive controller 260 can determine whether or not to bend the bending portion 112 beyond the maximum value of the bending angle of the bending portion 112 by comparing the traction quantity of the bending wire 160 with the traction quantity limit value MA. For example, it is possible to prevent the deformation of the joint ring (bending piece) 115 and the failure of the bending portion 112 due to the excessive bending of the bending portion 112.

It is desirable to set the traction quantity limit value MA in a shape of the flexible portion (a portion including the insertion portion 110 and the extracorporeal flexible portion 140) in which the bending angle of the bending portion 112 tends to increase with respect to the traction quantity of the bending wire 160 so that the traction quantity limit value MA becomes a value corresponding to the maximum value of the bending angle of the bending portion 112. The shape of the flexible portion in which the bending angle of the bending portion 112 tends to increase with respect to the traction quantity of the bending wire 160 is a linear shape (a first shape).

When the traction quantity of the bending wire 160 in the case where the bending wire 160 is driven on the basis of the bending manipulation exceeds the traction quantity limit value MA, the drive controller 260 may provide a first notification indicating that the drive of the bending wire 160 is limited to the practitioner S. The first notification is, for example, the display of a warning message on the display device 900 or the like.

### Step S140

The drive controller 260 acquires the tension of the bending wire 160 from the tension sensor 159 in step S140. In addition, the drive controller 260 may estimate the tension of the bending wire 160 from a torque acquired from the torque sensor (the first torque sensor 256e or the second torque sensor 257e) of the drive device 200 or a motor-specific electric current value of the motor portion (the first motor portion 256b or the second motor portion 257b). Subsequently, the drive controller 260 executes step S150.

### Step S150

In step S150, the drive controller 260 calculates the tension of the bending wire 160 when the bending wire 160 is driven on the basis of the received bending manipulation using the acquired tension of the bending wire 160 as a reference. The drive controller 260 compares the tension of the bending wire 160 when the bending wire 160 is driven on the basis of the received bending manipulation with the tension limit value MT.

The “tension limit value MT” is a value determined on the basis of the traction quantity of the bending wire 160. The “tension limit value MT” is greater than the tension of the bending wire 160 when no external force is applied to the distal end portion 111 and is a value less than the tension that breaks the bending portion 112.

The tension limit value MT is basically a value that increases as the traction quantity of the bending wire 160 increases. The tension limit value MT is, for example, a linear function, a quadratic function, a step function that increases step by step on the basis of a traction quantity or the like.

For example, the tension limit value MT is a linear function having a slope a and an intercept b. The slope a is calculated, for example, on the basis of Eq. (1). In Eq. (1), a denotes a constant (α≥ 1), C denotes a constant (C≥0), L denotes a length of the flexible portion (a portion including the insertion portion 110 and the external flexible portion 140) through which the bending wire 160 is inserted, and EA denotes the rigidity of the bending wire 160 through which the flexible portion is inserted.

The slope a is set with reference to a tension change k per traction quantity at no load. The tension change k is proportional to EA and inversely proportional to L. The larger the traction quantity of the bending wire 160, the greater the tension of the bending wire 160 and the greater the variation. Therefore, in the calculation of the slope a, the constant α and the constant C are provided so that the slope a is larger than the tension change k. Thereby, variations that tend to occur as the amount of traction increases can be tolerated.

$${a}_{L , EA} = α \frac{EA}{L} + C \qquad ( 1 )$$

The slope a and the intercept b may be calculated on the basis of Eq. (2) in consideration of the shape of the flexible portion (the portion including the insertion portion 110 and the extracorporeal flexible portion 140). In Eq. (2), D denotes a constant (D≥0), μ denotes a coefficient of friction between the bending wire 160 and the wire sheath 161s, and θ denotes a total bending angle of the flexible portion. αdenotes an increasing function of θ.

If the flexible portion is bent, the tension change k per traction quantity at no load increases. Therefore, α is increased according to the total bending angle θ of the flexible portion. The intercept b is set on the basis of the tension in a range that the bending portion 112 is not broken. When the flexible portion is bent, the force transmission rate to the bending portion 112 is decremented, but the tension limit value MT is incremented by the decrement.

$${a}_{L , EA} ( θ ) = α ( θ ) \frac{EA}{L} + C \qquad ( 2 )$$

$$b ( θ ) = {De}^{μθ}$$

When the tension of the bending wire 160 in the case where the bending wire 160 is driven on the basis of the received bending manipulation exceeds the tension limit value MT, the drive controller 260 does not drive the bending wire 160 and executes step S170. That is, the drive controller 260 drives the bending wire 160 so that the tension of the bending wire 160 does not exceed the tension limit value MT.

By comparing the tension of the bending wire 160 with the tension limit value MT, the drive controller 260 can determine whether or not the tension of the bending wire 160 is increasing due to a factor other than the bending drive of the bending portion 112 (for example, an external force is applied to the distal end portion 111).

It is desirable to set the tension limit value MT in a shape of a flexible portion (a portion including the insertion portion 110 and the extracorporeal flexible portion 140) in which the tension of the bending wire 160 tends to increase with respect to the traction quantity of the bending wire 160. The shape of the flexible portion in which the tension of the bending wire 160 tends to increase with respect to the traction quantity of the bending wire 160 is, for example, a bending shape (a second shape) such as a shape in which the flexible portion is bent by 90 degrees or more.

When the tension of the bending wire 160 in the case where the bending wire 160 is driven on the basis of the bending manipulation exceeds the tension limit value MT, the drive controller 260 may provide a second notification indicating that the drive of the bending wire 160 is limited to the practitioner S. The second notification is, for example, the display of a warning message on the display device 900 or the like. By distinguishing whether the notification is the first notification or the second notification, the practitioner can identify the cause of the limited drive of the bending wire 160.

### Step S160

The drive controller 260 drives the bending wire 160 on the basis of the received bending manipulation in step S160.

### Step S170

The drive controller 260 determines the end of the bending drive control in step S170. When the drive controller 260 determines not to end the bending drive control, step S110 is executed. When it is determined that the bending drive control is ended, the drive controller 260 executes step S180 to end the bending drive control.

The above-described bending drive control is performed for each of the four bending wires 160 (the upper bending wire 161u, the lower bending wire 161d, the left bending wire 1611, and the right bending wire 161r).

In addition, the bending drive control may be performed by the main controller 560 (mainly, the processor 561) controlling the wire drive unit (actuator) 250.

According to the electric endoscope system 1000 according to the present embodiment, the bending drive of the bending portion 112 is limited to a predetermined range and the maximum bending angle of the bending portion 112 is unlikely to decrease. By comparing the traction quantity of the bending wire 160 with the traction quantity limit value MA, it is possible to reliably prevent the bending portion 112 from bending beyond the maximum value of the bending angle. Moreover, the tension limit value MT corresponding to the traction quantity of the bending wire 160 is compared with the tension of the bending wire 160, such that the bending portion 112 can be bent within a range in which no external force is applied to the distal end portion 111 and the maximum bending angle of the bending portion 112 is not easily reduced.

Although the first embodiment of the present disclosure has been described above in detail with reference to the drawings, a specific configuration is not limited to this embodiment and design changes and the like may be included without departing from the spirit and scope of the present disclosure. Moreover, constituent elements shown in the above-described embodiment and modified examples can be appropriately combined and configured.

### Modified Example 1

FIG. 16 is a graph showing a modified example of the tension limit value MT.

When the traction quantity of the bending wire 160 is less than or equal to a threshold value Th, the tension limit value MT may be constant. When the traction quantity of the bending wire 160 is greater than the threshold value Th, the tension limit value MT increases as the traction quantity increases. In the actual procedure, the practitioner S may intentionally press the lumen with the bending portion 112. Therefore, in a range in which the bending angle is small (a range in which the traction quantity is small), a lower limit may be set on the tension limit value MT so that a certain amount of load can be applied to the bending portion 112.

### Modified Example 2

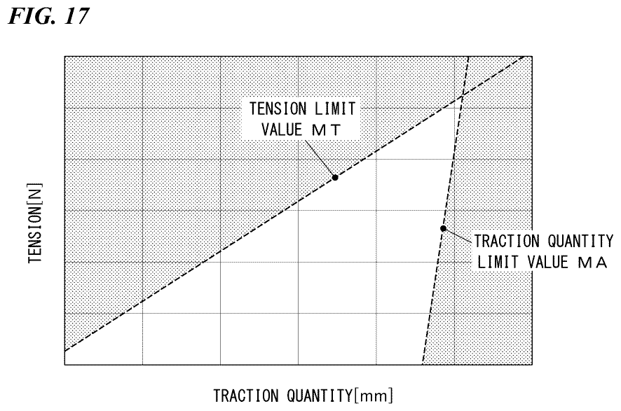

FIG. 17 is a graph showing a modified example of the traction quantity limit value MA.

The traction quantity limit value MA is not limited to a predetermined fixed value. The traction quantity limit value MA may be a value determined on the basis of the tension of the bending wire 160. The traction quantity limit value MA shown in FIG. 17 is a linear function that increases as the tension increases or the like.

A program in each embodiment may be recorded on a computer-readable recording medium and the program recorded on the recording medium may be read and executed by a computer system. The “computer system” used here is assumed to include an operating system (OS) or hardware such as peripheral devices. Moreover, the “computer-readable recording medium” refers to a flexible disk, a magneto-optical disc, a read-only memory (ROM), a portable medium such as a compact disc-ROM (CD-ROM), or a storage device such as a hard disk embedded in the computer system. Furthermore, the “computer-readable recording medium” may include a computer-readable recording medium for dynamically holding the program for a short time period as in a communication line when the program is transmitted via a network such as the Internet or a communication circuit such as a telephone circuit and a computer-readable recording medium for holding the program for a given time period as in a volatile memory inside the computer system serving as a server or a client when the program is transmitted. Moreover, the above-described program may be a program for implementing some of the above-described functions. Furthermore, the above-described program may be a program capable of implementing the above-described function in combination with a program already recorded on the computer system.

The present disclosure can be applied to a medical system for observing and treating the inside of a luminal organ or the like.

What is claimed is:

1. A medical manipulator system comprising:
a medical manipulator including a bending portion and a bending wire configured to bend the bending portion;
an actuator configured to bend the bending portion by driving the bending wire;
a sensor configured to detect tension of the bending wire; and
a controller configured to control the actuator,
wherein the controller drives the bending wire so that the tension of the bending wire does not exceed a tension limit value determined on the basis of a traction quantity of the bending wire.

2. The medical manipulator system according to claim 1, wherein the controller drives the bending wire so that the traction quantity of the bending wire does not exceed a predetermined traction quantity limit value.

3. The medical manipulator system according to claim 1, wherein the tension limit value increases as the traction quantity increases.

4. The medical manipulator system according to claim 1,
wherein the tension limit value is constant when the traction quantity of the bending wire is less than or equal to a threshold value, and
wherein the tension limit value increases as the traction quantity increases when the traction quantity of the bending wire is greater than the threshold value.

5. The medical manipulator system according to claim 1,
wherein the controller drives the bending wire so that the traction quantity of the bending wire does not exceed a traction quantity limit value determined on the basis of the tension of the bending wire, and
wherein the traction quantity limit value increases as the tension increases.

6. The medical manipulator system according to claim 1,
wherein the actuator includes an encoder attached to a drive axis of the bending wire, and
wherein the controller acquires the traction quantity of the bending wire from the encoder.

7. The medical manipulator system according to claim 2,
wherein the controller provides a first notification when the traction quantity of the bending wire exceeds the traction quantity limit value, and
wherein the controller provides a second notification different from the first notification when the tension of the bending wire exceeds the tension limit value.

8. A medical manipulator control method of controlling a bending operation of a medical manipulator, the medical manipulator control method comprising:
driving a bending wire so that tension of the bending wire that bends a bending portion of the medical manipulator does not exceed a tension limit value determined on the basis of a traction quantity of the bending wire.

9. The medical manipulator control method according to claim 8, wherein the bending wire is driven so that the traction quantity of the bending wire does not exceed a predetermined traction quantity limit value.

10. The medical manipulator control method according to claim 8, wherein the tension limit value increases as the traction quantity increases.

11. The medical manipulator control method according to claim 8,
wherein the tension limit value is constant when the traction quantity of the bending wire is less than or equal to a threshold value, and
wherein the tension limit value increases as the traction quantity increases when the traction quantity of the bending wire is greater than the threshold value.

12. The medical manipulator control method according to claim 8,
wherein the bending wire is driven so that the traction quantity of the bending wire does not exceed a traction quantity limit value determined on the basis of the tension of the bending wire, and
wherein the traction quantity limit value increases as the tension increases.

13. The medical manipulator control method according to claim 8, wherein the traction quantity of the bending wire is acquired from an encoder attached to a drive axis of the bending wire.

14. The medical manipulator control method according to claim 9,
wherein a first notification is provided when the traction quantity of the bending wire exceeds the traction quantity limit value, and
wherein a second notification different from the first notification is provided when the tension of the bending wire exceeds the tension limit value.

15. A control device for controlling a bending operation of a medical manipulator, the control device comprising:
driving a bending wire so that tension of the bending wire that bends a bending portion of the medical manipulator does not exceed a tension limit value determined on the basis of a traction quantity of the bending wire.

16. The control device according to claim 15, wherein the bending wire is driven so that the traction quantity of the bending wire does not exceed a predetermined traction quantity limit value.

17. The control device according to claim 15, wherein the tension limit value increases as the traction quantity increases.

18. The control device according to claim 15,
wherein the tension limit value is constant when the traction quantity of the bending wire is less than or equal to a threshold value, and
wherein the tension limit value increases as the traction quantity increases when the traction quantity of the bending wire is greater than the threshold value.

19. The control device according to claim 15,
wherein the bending wire is driven so that the traction quantity of the bending wire does not exceed a traction quantity limit value determined on the basis of the tension of the bending wire, and
wherein the traction quantity limit value increases as the tension increases.

20. The control device according to claim 15, wherein the traction quantity of the bending wire is acquired from an encoder attached to a drive axis of the bending wire.
